(19) United States
(12) Patent Application Publication
(10) Pub. No.: US 2024/0059722 A1
(43) Pub. Date: Feb. 22, 2024

(54) COORDINATION COMPLEX COMPOUND-ZIF COMPLEX AND METHOD OF PREPARING SAME

(71) Applicant: KOREA RESEARCH INSTITUTE OF CHEMICAL TECHNOLOGY, Daejeon (KR)

(72) Inventors: Young Kyu HWANG, Daejeon (KR); Chang Ho YOO, Daejeon (KR); Kyung Ryul OH, Daejeon (KR); Dong Won HWANG, Daejeon (KR); Gwang Nam YUN, Daejeon (KR); Mi Jung LEE, Daejeon (KR)

(73) Assignee: KOREA RESEARCH INSTITUTE OF CHEMICAL TECHNOLOGY, Daejeon (KR)

(21) Appl. No.: 18/450,131

(22) Filed: Aug. 15, 2023

(30) Foreign Application Priority Data
Aug. 17, 2022 (KR) 10-2022-0102520

(51) Int. Cl. C07F 15/00; B01J 23/46

(52) U.S. Cl. CPC C07F 15/0033; B01J 23/468

(57) ABSTRACT

Proposed are a coordination complex compound-ZIF complex and a method of preparing the same. More particularly, proposed are a coordination complex compound-ZIF complex, which is a complex on which a coordination complex compound is supported by being chemically and directly bonded to a metal vacancy site of a ZIF, capable of being usable as a heterogeneous catalyst that can be recovered and reused in subsequent processes and maintaining catalytic functions of the coordination complex compound, which is an active ingredient, and to a method of preparing the same.

### CROSS REFERENCE TO RELATED APPLICATION

The present application claims priority to Korean Patent Application No. 10-2022-0102520, filed Aug. 17, 2022, the entire contents of which is incorporated herein for all purposes by this reference.

### BACKGROUND OF THE DISCLOSURE

### 1. Field of the Disclosure

The present disclosure relates to a coordination complex compound-zeolitic imidazolate framework (ZIF) complex and a method of preparing the same. More particularly, the present disclosure relates to a coordination complex compound-ZIF complex, which is a complex on which a coordination complex compound is supported by being chemically and directly bonded to a metal vacancy site of a ZIF, capable of being usable as a heterogeneous catalyst that can be recovered and reused in subsequent processes and maintaining catalytic functions of the coordination complex compound, which is an active ingredient, and to a method of preparing the same.

### 2. Description of the Related Art

A coordination complex compound, produced by the coordination bonding of a ligand to a central metal, mainly involves a chemical bond between carbon and metal but sometimes also includes an organometallic compound having a metal-nitrogen or metal-phosphorus bond. The coordination complex compound acts as a catalyst and can have a wide range of applications in many industrial fields, including the production of petrochemical products, the production of organic polymers, the production of polymers, and the like. The field of application varies depending on the type of central metal, oxidation number, electron density, type of ligand, and coordination structure, and the catalytic activity also varies greatly. In the case of a liquid-phase reaction, a coordination complex compound dissolves in a solvent in the form of molecular units and thus is classified as a homogeneous catalyst, which exhibits extremely high activity compared to heterogeneous catalysts that do not dissolve in a solvent and react only on the metal surface.

As described above, even though a coordination complex compound exhibits high activity when being used as a catalyst, recovery and reuse after catalysis are extremely difficult when being used as a homogeneous catalyst. Attempts have been made to recover the coordination complex compound by evaporating a product solution with heat application for reuse. However, in this case, the product may change in composition due to additional reactions, and the coordination complex compound may also be deformed. In particular, in the case of using expensive precious metal as a central metal, when the catalyst is difficult to be recovered, there is a limit for use as an industrial catalyst due to the expansive manufacturing costs.

On the other hand, a zeolitic imidazolate framework (ZIF), one type of porous metal-organic framework (hereinafter referred to as MOF), contains Zn or Co having an oxidation state of +2 as a central metal and is a material that forms a coordination bond with an imidazolate ligand in a tetrahedral structure to form a three-dimensional structure similar to zeolite. ZIFs, like MOFs, have a large surface area, various pore sizes, and a plurality of metal or linker vacancy sites. In addition, ZIFs have higher thermal and chemical stability than MOFs, thus being used in various fields, and can also be used as a carrier for catalysts.

There has been an attempt to synthesize a multi-metal ZIF in which a part of a central metal of an existing ZIF is substituted with another metal ion for use as a catalyst. In Non-Patent Document 001, a multi-metal ZIF, a carbon catalyst containing monoatomic metal by performing additional heat treatment, in which a tetrahedrally coordinated structure of imidazolate of the ZIF formed around metal introduced into the ZIF exhibits activity, is used for oxygen reduction reaction (ORR) in the field of electrochemistry.

However, the multi-metal ZIF having a form in which a part of the central metal of the ZIF is substituted with another metal ion forms the tetrahedrally coordinated structure of the imidazolate of the ZIF formed around the metal introduced into the ZIF. Thus, there is no space for reactants to be adsorbed other than a ligand vacancy site. For this reason, there is a problem in that catalysis is severely limited.

To overcome the problem described above, a method of substituting not only the metal ion but also imidazolate of the ZIF with other ligands may be taken into account. However, a stable metal-imidazolate bond is not easy to be substituted, and such an attempt is yet to be published.

A method of introducing a coordination complex compound having a form in which a metal ion is bonded to an organic ligand into a vacancy site of a ZIF may be taken into account as an alternative method, instead of introducing a monoatomic metal ion or ligand. According to the 18-electron rule, a metal ion alone theoretically is difficult to be coordinated with imidazolate at a metal vacancy site of a ZIF. However, a coordination complex compound in which 18 electrons are maintained by bonding a central metal to an organic ligand may be stably coordinated with imidazolate. Given that coordination complex compounds with excellent catalytic activity are available and recovery and reuse of the coordination complex compounds are easy at the same time, a technology for introducing such a coordination complex compound into the metal vacancy site of a ZIF is required to be secured.

However, coordination complex compounds with a large molecular size typically have a problem in that steric strain occurs in the process of reaching a metal vacancy site of a ZIF. Furthermore, in using heterogeneous catalyst complexes in which a coordination complex compound is introduced into the ZIF, the above problem remained a challenge in the current state of the art.

### DOCUMENT OF RELATED ART

### Patent Document

(Patent Document 001) CN 111454462 (Jul. 28, 2020)

### Non-Patent Document

(Non-Patent Document 001) Q. Liu et al., Adv. Energy. Mater. 2020, 2000689

### SUMMARY OF THE INVENTION

An objective of the present disclosure is to provide a coordination complex compound-zeolitic imidazolate framework (ZIF) complex in which a coordination complex compound and a ZIF are bonded.

In addition, another objective of the present disclosure is to provide a method of preparing a coordination complex compound-ZIF complex in which a coordination complex compound is introduced into a metal vacancy site of a ZIF.

To solve the problems described above, the present disclosure provides a method of preparing a coordination complex compound-ZIF complex, the method characterized by including: (a) preparing a coordination complex compound precursor containing both of a ligand more strongly bonded to a central metal of a coordination complex compound than imidazolate and a ligand more weakly bonded to the central metal of the coordination complex compound than imidazolate; and (b) obtaining a coordination complex compound-ZIF complex by adding the coordination complex compound precursor and a ZIF to a basic solution and heating the resulting solution to a temperature in a range of 5° C. to 250° C.

In addition, the present disclosure provides a method of preparing a coordination complex compound-ZIF complex, the method characterized by including: (A) preparing a coordination complex compound precursor and a composition for synthesizing a ZIF, the coordination complex compound precursor containing both of a ligand more strongly bonded to a central metal of a coordination complex compound than imidazolate and a ligand more weakly bonded to the central metal of the coordination complex compound than imidazolate; and (B) obtaining a coordination complex compound-ZIF complex by heating a mixed solution of the coordination complex compound precursor, the ZIF, and a base to a temperature in a range of 5° C. to 250° C.

The ZIF may be one of ZIF-11, ZIF-1, ZIF-4, ZIF-7, ZIF-8, ZIF-9, ZIF-12, ZIF-35, ZIF-67, ZIF-69, ZIF-71, ZIF-90, ZIF-95, and ZIF-100.

The coordination complex compound may be added in an amount in a range of 0.01 to 10 mol % with respect to the number of moles of Zn contained in the ZIF.

In addition, the present disclosure provides a complex in which a coordination complex compound is bonded to a ZIF, the complex characterized in that a central metal of the coordination complex compound is chemically and directly bonded to a metal vacancy site of the ZIF.

A ligand of the coordination complex compound may be characterized by being more strongly bonded to the central metal of the coordination complex compound than imidazolate, thereby not being substituted by the imidazolate.

The ZIF may be one of ZIF-11, ZIF-1, ZIF-4, ZIF-7, ZIF-8, ZIF-9, ZIF-12, ZIF-35, ZIF-67, ZIF-69, ZIF-71, ZIF-90, ZIF-95, and ZIF-100.

The central metal of the coordination complex compound may include at least one of iridium (Ir), cobalt (Co), iron (Fe), ruthenium (Ru), rhodium (Rh), platinum (Pt), palladium (Pd), gold (Au), silver (Ag), copper (Cu), nickel (Ni), titanium (Ti), zirconium (Zr), chromium (Cr), molybdenum (Mo), and rhenium (Re).

A ligand of the coordination complex compound may include at least one of carbene, phosphine, amine, and cyclopentadienyl.

In addition, the coordination complex compound-ZIF complex of the present disclosure may be characterized by being represented by Formula 1.

M₁₋ₓ(L)₂(MC)ₓ [Formula 1]

In Formula 1, M is the central metal of the ZIF and is selected from among Zn, Co, Ni, Fe, Mn and Pd, L is imidazolate, MC is the coordination complex compound chemically bonded to a metal vacancy site of the ZIF, where a ligand bonded to the central metal of the coordination complex compound is more strongly bonded to the central metal of the coordination complex compound than imidazolate, thereby being unsubstituted by imidazolate, and x is a real number greater than 0 and less than or equal to 0.1

In addition, the present disclosure provides a method of obtaining a conversion product by transfer-hydrogenation of a hydrocarbon containing at least one OH group, such as glycerol and the like, or a method of obtaining formic acid by converting carbon dioxide, using a catalyst containing the coordination complex compound-ZIF complex.

According to the present disclosure, a coordination complex compound-ZIF complex in which a metal of the coordination complex compound is directly and chemically bonded to a metal vacancy site of a ZIF can be prepared.

In addition, the coordination complex compound-ZIF complex of the present disclosure is advantageous that the coordination complex compound is introduced into the ZIF to be converted into a heterogeneous catalyst form, thereby maintaining high activity and being easily recovered and reused after the reaction at the same time.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates diagrams of examples of a ZIF having metal vacancy sites and linker vacancy sites;

FIG. 1B illustrates diagrams of examples of a central metal of a coordination complex compound being chemically and directly bonded to metal vacancy sites of a ZIF;

FIG. 2A illustrates structure of Ir-NHC catalyst as an example of coordination complex compounds of the present disclosure;

FIG. 2B illustrates structure of Ir-NHC2 catalyst as an example of coordination complex compounds of the present disclosure;

FIG. 2C illustrates structure of Ir-Cp* catalyst as an example of coordination complex compounds of the present disclosure;

FIG. 3 illustrates a diagram of a method of preparing ZIFs or Ir-NHC of comparative examples of the present disclosure and coordination complex compound-ZIF complexes of examples of the present disclosure;

FIG. 4A shows diagrams of EDS mapping results of coordination complex compound-ZIF complexes INZ-A of the present disclosure;

FIG. 4B shows diagrams of EDS mapping results of coordination complex compound-ZIF complexes INZ-B of the present disclosure;

FIG. 5A shows graphs of turnover number (TON) when using coordination complex compound-ZIF complexes of the present disclosure as a catalyst for glycerol dehydrogenation;

FIG. 5B shows graphs of turnover number (TON) when using coordination complex compound-ZIF complexes of the present disclosure as a catalyst for CO₂ hydrogenation;

FIG. 6 shows a graph of changes in the number of moles of FA produced according to the reaction time of CO₂ hydrogenation before and after performing high-temperature filtration of an INZ-A complex catalyst of the present disclosure;

FIG. 7A shows graphs of turnover number (TON) measurement results according to the number of repeated use of an INZ-A complex catalyst of the present disclosure in K₂CO₃-mediated transfer-hydrogenation of glycerol;

FIG. 7B shows graphs of XRD measurement results according to the number of repeated use of an INZ-A complex catalyst of the present disclosure in K₂CO₃-mediated transfer-hydrogenation of glycerol; and

FIG. 8 illustrates a coordination structure of a central metal Ir of an INZ-A complex.

### DESCRIPTION OF THE PREFERRED EMBODIMENTS

Unless defined otherwise, all technical and scientific terms used herein have the same meaning as commonly understood by those skilled in the art to which the present disclosure belongs. In general, the nomenclature used herein is well-known and commonly used in the art. Unless the context clearly indicates otherwise, it will be further understood that the terms “comprises”, “comprising,”, “includes”, and/or “including”, when used herein, specify the presence of stated features, integers, steps, operations, elements, and/or components, but do not preclude the presence or addition of one or more other features, integers, steps, operations, elements, components, and/or groups thereof.

In the technical field to which the present disclosure belongs, most coordination complex compounds with excellent catalytic activity are homogeneous catalysts. Thus, there is a problem in that separation and purification processes from a reaction solvent are required to be involved after the reaction.

Even though a method of immobilizing nanoclusters in ZIF pores has been disclosed as a solution to the above problem, there have been no reported cases of immobilizing a coordination complex compound as a catalyst.

As another solution to the above problem, there is a method of converting a homogeneous catalyst into a heterogeneous catalyst through support and the like. However, coordination complex compounds have a relatively larger size than metal atoms, so the introduction of a coordination complex compound itself into a metal vacancy site of a ZIF was difficult to be realized.

To solve the problems described above, the applicants of the present disclosure have discovered that a coordination complex compound-ZIF complex in which a central metal of the coordination complex compound is chemically and directly bonded to a metal vacancy site of a ZIF is able to be prepared using a coordination complex compound precursor containing two types of ligands, a first ligand that is chemically and strongly bonded to the central metal ion and a second ligand that is easily dissociated, by a method in which a part of the ligand in the precursor is dissociated and introduced into the ZIF metal vacancy site, thereby completing the present disclosure.

The coordination complex compound-ZIF complex prepared by the preparation method of the applicants of the present disclosure is a type of single-metal catalyst in which metal is dispersed in atomic units and thus has great material and research values. In addition, the coordination complex compound-ZIF complex enables excellent catalytic activity of the coordination complex compound to be maintained and has an effect of being easily recovered and reused as a heterogeneous catalyst at the same time, which is industrially applicable.

Hereinafter, the coordination complex compound-ZIF complex of the present disclosure and a method of preparing the same will be described.

The present disclosure relates to a complex in which a coordination complex compound is bonded to a ZIF and is characterized in that a central metal of the coordination complex compound is chemically and directly bonded to and supported on a metal vacancy site of the ZIF. The zeolitic imidazolate framework (ZIF) is one type of metal-organic framework (MOF). The metal-organic framework, a microporous crystalline material composed of metal atoms or metal clusters and organic linkers connecting them through a coordination bond, is a relatively new hybrid organic-inorganic material. The ZIF is composed of a metal ion (typically, zinc or cobalt) linked to an imidazolate (or imidazolate derivative) ligand. The ZIF has a metal-linker-metal bonding angle that is similar to the Si—O—Si bonding angle found in many zeolites but has distinct differences in their constituent elements. Therefore, such ZIFs have attracted attention due to the excellent thermal and chemical stability thereof with ultrafine porosity, and have a wide range of industrial applications. Typically, a ZIF is prepared by bonding a metal ion serving as a central metal selected from among Zn, Co, Ni, Fe, Mn and Pd, and an imidazole derivative serving as a ligand that is unsubstituted with a functional group other than hydrogen so that nitrogen atoms present at the first and third positions of imidazole ring are bonded to the metal ion.

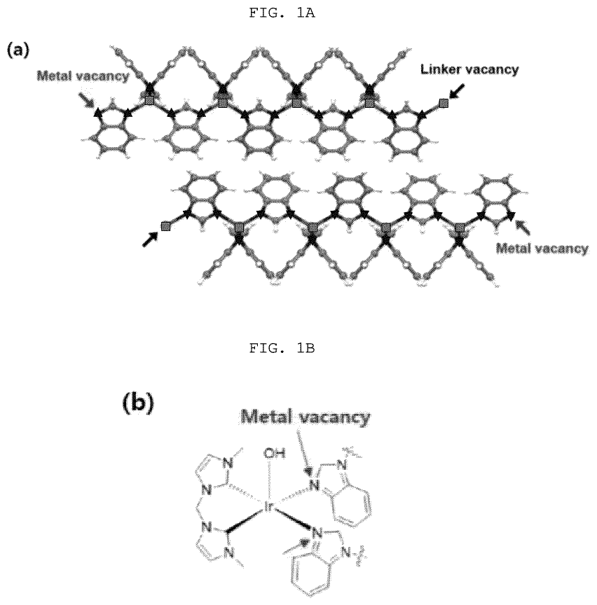

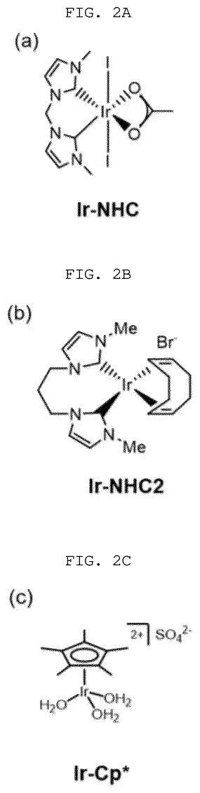

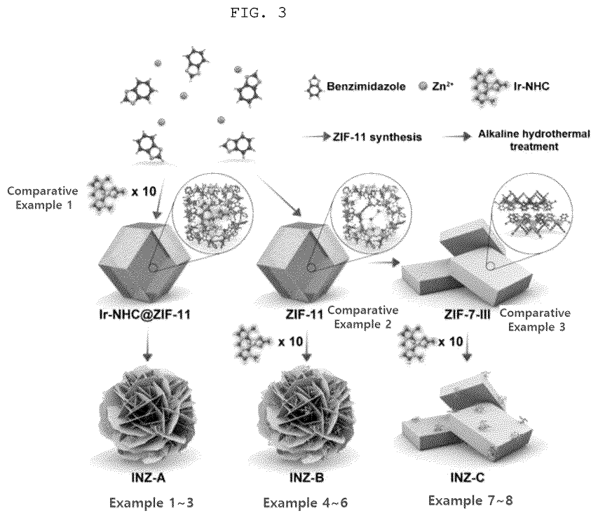

FIG. 1A illustrates diagrams of examples of a ZIF having metal vacancy sites and linker vacancy sites; and FIG. 1B illustrates diagrams of examples of a central metal of a coordination complex compound being chemically and directly bonded to metal vacancy sites of a ZIF.

Referring to FIG. 1A and FIG. 1B, the central metal of the coordination complex compound being chemically and directly bonded to and supported on the metal vacancy site of the ZIF means that while there is a metal vacancy site where the central metal bonded to a nitrogen functional group (—N group) in an imidazolate heterocycle of the originally synthesized ZIF is detached, the central metal of the coordination complex compound is bonded to the metal vacancy site. This is a technical feature of the complex of the present disclosure, which is distinguished from that in which the ligand of the coordination complex compound is directly bonded or is bonded through a linker.

The complex of the present disclosure, which has the technical feature in its unique bonding site between the coordination complex compound and the ZIF of the present disclosure, may be used for various purposes, including sensors, adsorbents, catalysts, and the like.

A coordination complex compound narrowly means a compound in which carbon and a metal are chemically bonded to each other. However, in the present disclosure, the coordination complex compound is more broadly defined as a compound containing a chemical bonding between metal and one of carbon, phosphorus, and nitrogen,

The coordination complex compound contains the central metal and the ligand linked thereto as catalyst components. The central metal may be at least one of iridium (Ir), cobalt (Co), iron (Fe), ruthenium (Ru), rhodium (Rh), platinum (Pt), palladium (Pd), gold (Au), silver (Ag), copper (Cu), nickel (Ni), titanium (Ti), zirconium (Zr), chromium (Cr), molybdenum (Mo), and rhenium (Re). Preferably, the central metal is at least one of iridium (Ir), cobalt (Co), and iron (Fe).

In addition, the coordination complex compound is characterized by containing a ligand more strongly bonded to the central metal of the coordination complex compound than imidazolate.

Specifically, the coordination complex compound bonded to the complex is derived from a coordination complex compound precursor. On one side of the coordination complex compound precursor, both of the ligand more strongly bonded to the central metal of the coordination complex compound than imidazolate and a ligand more weakly bonded to the central metal of the coordination complex compound than imidazolate are bonded. The ligand more weakly bonded to the central metal of the coordination complex compound than imidazolate, which is anionic, is dissociated from the central metal in a ZIF-containing alkaline environment, and the dissociation site becomes a bonding site with the metal vacancy site of the ZIF.

Whether the ligand is more strongly or weakly bonded to the central metal of the coordination complex compound than imidazolate may be predicted on the basis of pKa values. In most cases, when the conjugate acid of the ligand has a pKa value higher than 14, which is the pKa value of imidazole (the conjugate acid of imidazolate), the ligand is more strongly bonded to the central metal of the coordination complex compound than imidazolate, thereby being unsubstituted by the imidazolate. On the contrary, when the conjugate acid of the ligand has a pKa value lower than 14, which is the pKa value of imidazole, the ligand is more weakly bonded to the central metal of the coordination complex compound than imidazolate, thereby being substituted by the imidazolate.

The ligand more strongly bonded to the central metal of the coordination complex compound than imidazolate may be at least one of carbene, phosphine, amine, and cyclopentadienyl.

The carbene is a compound containing a carbene carbon atom, which is a divalent carbon atom having 6 electrons in the outermost electron shell and has a formula of CR₁R₂, where R₁ and R₂ are the same as or different from each other. In addition, R₁ and R₂ may be each independently selected from among hydrogen, deuterium, an aliphatic hydrocarbon having 1 to 30 carbon atoms, an alicyclic hydrocarbon having 3 to 30 carbon atoms, an alicyclic heterocyclic hydrocarbon having 3 to 30 carbon atoms, an aromatic hydrocarbon having 5 to 30 carbon atoms, and an aromatic heterocyclic hydrocarbon having 5 to 30 carbon atoms. Furthermore, R₁ and R₂ may be linked to each other to additionally form an alicyclic or aromatic monocyclic, polycyclic, or heterocyclic ring. For example, at least one of cyclohexane, benzene, naphthalene, anthracene, phenanthrene, chrysene, and N-heterocyclic carbene may be used without limitation. Preferably, the carbene includes N-heterocyclic carbene (hereinafter referred to as NHC).

The NHC is a heterocyclic compound containing a carbene carbon atom and one or more nitrogen atoms in a ring, and does not satisfy the octet rule of the outermost electron shell, thereby being highly reactive. Highly reactive NHC is a ligand more strongly bonded to the central metal of the coordination complex compound than imidazolate. At least one among imidazolylidene, imidazolinylidene, thiazolylidene, oxazolylidene, triazolylidene, benzimidazolylidene, pyrrolidinylidene, and N,N′-diamidocarbene may be used without limitation. Preferably, imidazolylidene is used, and more preferably, bis-imidazolylidene linked by an alkyl group having 1 to 3 carbon atoms is used.

The phosphine has a formula of PR₃R₄R₅, where R₃, R₄, and R₅ are the same as or different from each other. In addition, R₃, R₄, and R₅ may be each independently selected from among hydrogen, deuterium, an aliphatic hydrocarbon having 1 to 30 carbon atoms, an alicyclic hydrocarbon having 3 to 30 carbon atoms, and an aromatic hydrocarbon having 5 to 30 carbon atoms. Furthermore, two of R₃, R₄, and R₅ may be linked to each other to additionally form an alicyclic or aromatic monocyclic or polycyclic ring. For example, at least one of phosphole, phosphinine, phosphanaphthalene, and phosphaphenalene may be used without limitation, and at least one compound in the form of mono-phosphine, bis-phosphine, and tris-phosphine may also be used.

The amine has a formula of NR₆R₇R₈, where R₆, R₇, and R₈ are the same as or different from each other. In addition, R₆, R₇, and R₈ may be each independently selected from among hydrogen, deuterium, an aliphatic hydrocarbon having 1 to 30 carbon atoms, an alicyclic hydrocarbon having 3 to 30 carbon atoms, and an aromatic hydrocarbon having 5 to 30 carbon atoms. Furthermore, any two of R R₆, R₇, and R₈ may be linked to each other to additionally form an alicyclic or aromatic monocyclic or polycyclic ring. For example, at least one of pyrrole, pyridine, pyrimidine, imidazole, pyrrolidine, indole, piperidine, quinoline, pyrazole, and 1,2,4-triazole may be used without limitation, and at least one compound in the form of mono-amine, bis-amine, and tris-amine may also be used.

The cyclopentadienyl ligand contains the same or different substituent bonded to carbon atoms constituting the aromatic ring. In addition, the substituent may be selected from among hydrogen, deuterium, an aliphatic hydrocarbon having 1 to 30 carbon atoms, an alicyclic hydrocarbon having 3 to 30 carbon atoms, and an aromatic hydrocarbon having 5 to 30 carbon atoms. Furthermore, a ligand in which two cyclopentadienyl ligands are linked by carbon or silicon is included.

The zeolitic imidazolate framework (ZIF), one type of metal-organic framework (MOF) with high thermal and chemical stability, has a structure in which Zn or Co metal is bonded to imidazolate, which is a linker. In addition, the ZIF has a metal vacancy site free of Zn or Co metal and a linker vacancy site free of imidazolate. The ZIF, which is a porous material, is used as a catalyst support.

As the ZIF, one of ZIF-11, ZIF-1, ZIF-4, ZIF-7, ZIF-8, ZIF-9, ZIF-12, ZIF-35, ZIF-67, ZIF-69, ZIF-71, ZIF-90, ZIF-95, and ZIF-100 may be used. However, the ZIF is not limited to the types listed above.

In addition, the coordination complex compound-ZIF complex, according to the present disclosure, may be represented by Formula 1 below.

M₁₋ₓ(L)₂(MC)ₓ [Formula 1]

In this case, M is the central metal of the ZIF and is selected from among Zn, Co, Ni, Fe, Mn and Pd, L is imidazolate, MC is the coordination complex compound chemically bonded to the metal vacancy site of the ZIF, where the ligand bonded to the central metal of the coordination complex compound is more strongly bonded to the central metal of the coordination complex compound than imidazolate, thereby being unsubstituted by imidazolate, and x is a real number greater than 0 and less than or equal to 0.1.

Hereinafter, a method of preparing the coordination complex compound-ZIF complex according to the present disclosure will be described.

As one example of the preparation method of the present disclosure, a method of preparing the coordination complex compound-ZIF complex is characterized by including: (a) preparing a coordination complex compound precursor containing both of a ligand more strongly bonded to a central metal of the coordination complex compound than imidazolate and a ligand more weakly bonded to the central metal of the coordination complex compound than imidazolate, and (b) obtaining a coordination complex compound-ZIF complex by adding the coordination complex compound precursor and a ZIF to a basic solution and heating the resulting solution to a temperature in a range of 5° C. to 250° C.

The preparation method of the present disclosure, including the (a) preparing and the (b) obtaining, is characterized by including bonding the coordination complex compound to the synthesized ZIF.

The (a) preparing, which is a step of preparing the coordination complex compound precursor, may be a step of preparing a solution by dissolving the coordination complex compound precursor containing both of the ligand more strongly bonded to the central metal of the coordination complex compound than imidazolate and the ligand more weakly bonded to the central metal of the coordination complex compound than imidazolate in a solvent.

As described above with reference to the coordination complex compound-ZIF complex, the coordination complex compound precursor forms the coordination complex compound when the ligand more weakly bonded to the central metal of the coordination complex compound than imidazolate, bonded to one side of the coordination complex compound precursor, is dissociated.

In this case, the coordination complex compound may be prepared in a solution form by dissolving in the solvent. As the solvent, any solvent capable of dissolving the coordination complex compound may be used without limitation. However, at least one of toluene, benzene, xylene, dichloromethane, dichloroethane, tetrahydrofuran, chloroform, methanol, ethanol, propanol, butanol, and water is preferably used as the solvent.

The base may be at least one among hydroxide salts of alkali metals or alkaline earth metals, and ammonia, and with the addition of the base, the mixed solution may have a pH in a range of 8 to 14.

The (b) obtaining is a step of obtaining the coordination complex compound-ZIF complex by heating the mixed solution of the coordination complex compound precursor, the ZIF, and the base.

The ligand more weakly bonded to the central metal of the coordination complex compound than imidazolate, which is anionic, is dissociated in a ZIF-containing alkaline environment to form the coordination complex compound, and then is bonded to imidazolate of the ZIF by positioning the central atom of the coordination complex compound at the metal vacancy site of the ZIF.

The heating may be performed at a temperature in a range of 5° C. to 250° C. The heated coordination complex compound-ZIF complex may be additionally subjected to filtration, washing, and drying processes using known methods to obtain the coordination complex compound-ZIF complex.

The coordination complex compound may be added in an amount in a range of 0.01 to 10 mol % with respect to the number of moles of the central metal contained in the ZIF.

In addition, as another example of the preparation method of the present disclosure, a method of preparing the coordination complex compound-ZIF complex may be characterized by including: (A) preparing a coordination complex compound precursor and a composition for synthesizing ZIF, the coordination complex compound precursor containing both of a ligand more strongly bonded to a central metal of the coordination complex compound than imidazolate and a ligand more weakly bonded to the central metal of the coordination complex compound than imidazolate, and (B) obtaining a coordination complex compound-ZIF complex by heating a mixed solution of the coordination complex compound precursor, the ZIF, and a base to a temperature in a range of 5° C. to 250° C.

The preparation method of the present disclosure, including the (A) preparing and the (B) obtaining, is characterized by including introducing the coordination complex compound into ZIF synthesis.

In the (A) preparing, the composition for synthesizing the ZIF contains a precursor of a metal selected from among Zn, Co, Ni, Fe, Mn and Pd, benzimidazole as a precursor of imidazolate, and the like. Any composition known in the art for synthesizing the ZIF may be used.

Subsequently, the composition for synthesizing the ZIF and the coordination complex compound precursor are mixed in a solvent. Any solvent capable of dissolving the coordination complex compound may be used without limitation. However, at least one of toluene, benzene, xylene, dichloromethane, dichloroethane, tetrahydrofuran, chloroform, methanol, ethanol, propanol, butanol, and water is preferably used as the solvent.

In the (B) obtaining, the heating may be performed at a temperature in a range of 5° C. to 250° C. The heated coordinated complex compound-ZIF complex may be additionally subjected to filtration, washing, and drying processes using known methods to obtain the coordination complex compound-ZIF complex.

Hereinafter, the range of the number of moles of the coordination complex compound with respect to the number of moles of Zn contained in the ZIF is the same as that described above with reference to the preparation method of the complex, including the (a) preparing and (b) obtaining, so redundant descriptions will be omitted.

In addition, the present disclosure provides a method of producing formic acid by converting carbon dioxide using the coordination complex compound-ZIF complex as a catalyst. In the conversion reaction of carbon dioxide, hydrogen or a hydrocarbon containing at least one hydroxyl group may be included as a reactant other than carbon dioxide. Hydrogen or the hydrocarbon containing at least one hydroxyl group acts as a reducing material for reducing carbon dioxide through catalysis. The hydrocarbon containing at least one hydroxyl group first produces hydrogen through dehydrogenation. The produced hydrogen may act as a reactant for reducing carbon dioxide. Alternatively, hydrogen directly from the hydrocarbon containing at least one hydroxyl group may reduce carbon dioxide through a process of being directly transferred to carbon dioxide.

In the present disclosure, the term “hydrocarbon containing at least one hydroxyl group” may be one or a mixture selected from among monohydric alcohols and polyhydric alcohols, or a mixture thereof. The hydrocarbon containing at least one hydroxyl group may be one selected from the group consisting of glucose, maltose, galactose, xylose, sorbitol, mannitol, calactitol, xylitol, glycerol, 1,4-butanediol or an isomer thereof, 1,4-pentanediol or an isomer thereof, 1,2-propanediol or an isomer thereof, butanol, pentanol, propanol, and chitin derived compound which is preferable in terms of reactivity with carbon dioxide. From an environmental point of view as well as a raw material supply and demand point of view, the hydrocarbon containing at least one hydroxyl group is more preferably glucose, xylose, and glycerol. From an environmental point of view, the “hydrocarbon containing at least one hydroxyl group” is preferably derived from biomass.

In the reaction, the source of carbon dioxide may be gaseous carbon dioxide, but preferably has a form of an alkali metal carbonate, an alkali metal bicarbonate, an alkaline earth metal carbonate, or an alkaline earth metal bicarbonate formed by reacting carbon dioxide with an alkali metal hydroxide and an alkaline earth metal hydroxide.

In addition, the present disclosure provides a method of obtaining a conversion product by transfer-hydrogenation of the hydrocarbon containing at least one hydroxyl group through the transfer-hydrogenation of the hydrocarbon containing at least one hydroxyl group.

The conversion reaction of carbon dioxide or the transfer-hydrogenation of hydrocarbons containing at least one hydroxy group may be performed at a temperature in a range of room temperature to 300° C. at a pressure in a range of 1 to 200 bar, and is preferably performed at a temperature in a range of 100° C. to 250° C. at a pressure in a range of 20 to 120 bar.

Hereinafter, preferred embodiments of the coordination complex compound-ZIF complex of the present disclosure and the method of preparing the same will be described. For reference, although the following examples are provided to illustrate one or more preferred embodiments of the present disclosure, the present disclosure is not limited thereto. Various modifications can be made to the following examples falling within the scope of the present disclosure.

### <Comparative Example 1> Preparation of Ir^{III}-bis(NHC) (CH₃COO)I₂ (Ir-NHC)

A mixed reactant of 300 g (0.694 mmol) of Ligand A (3,3′-methylene-bis(1-methyl-1H-imidazol-3-ium)iodide), 233 mg (0.347 mmol) of [Ir(COD)Cl]₂ (cyclooctadiene iridium chloride dimer), 230 mg (1.39 mmol) of potassium iodide (KI), and 342 mg (2.78 mmol) of sodium acetate was added to 15 mL of 0.05 M acetonitrile and then stirred while being refluxed for 16 hours. The cooled reactant was concentrated using a rotary evaporator and then purified by column chromatography (CH₂Cl₂/acetone=8:2) to obtain Ir-NHC (yield: 40%).

### <Comparative Example 2> Preparation of Ir^{III}-bis(NHC) (COD)Br (Ir-NHC2)

44 mg (1.1 mmol) of sodium hydride dissolved in 6 mL of ethanol, and then the resulting solution was added to a solution in which 134 mg (0.2 mmol) of [Ir(COD)Cl]₂ dissolved in 6 mL of ethanol. After stirring the obtained mixed solution at room temperature for 1 hour, 183 mg (0.5 mmol) of Ligand B (1,1′-(propane-1,3-diyl)bis(3-methyl-imidazole-3-ium)dibromide) was added thereto and stirred at room temperature for 12 hours. The obtained mixed solution was filtered, and the obtained filtrate solution was dried in vacuo. The obtained solid powder dissolved in CH₂Cl₂ and then filtered through a syringe filter again. Then, the filtrate solution was dried in vacuo to obtain Ir^{III}-bis (NBC) (COD)Br (yield: 80%).

### <Comparative Example 3> Preparation of [Ir^{III}-Cp*(H₂O)₃]SO₄ (Ir-Cp*)

In a nitrogen-filled glovebox, 3.5 mmol of iridium trichloride hydrate dissolved in 50 mL of methanol in a 250-mL two-neck round-bottom flask, and then 4.83 mmol of pentamethylcyclopentadiene was slowly added thereto. The reaction solution obtained by undergoing a reaction for 24 hours in a nitrogen reflux system was filtered. Next, the filtered dark purple solid was washed three times with 10 mL of chloroform and dried in vacuo under the condition at room temperature to obtain a DiCp*-diiridium complex. Then, 1 mmol of the DiCp*-diiridium complex and 2 mmol of silver sulfate were added to 10 mL of distilled water and stirred at room temperature for 24 hours, followed by filtering and purifying the obtained solution. The filtered solution was evaporated using a rotary evaporator and dried in vacuo at room temperature to obtain yellow solid [(pentamethylcyclopentadienyl)iridium(III) (H₂O)₃] (SO₄) (Ir-Cp*) (yield: 73%).

### <Comparative Example 4> Preparation of ZIF-11

20 mmol of benzimidazole dissolved in 3 mol of methanol, and then 20 mmol of ammonia (using 30% ammonium hydroxide aqueous solution), following 1 mol of toluene, was added to prepare a composition for synthesizing ZIF-11. Subsequently, 10 mmol of a zinc acetate hydrate (Zn(CH₃COO)₂·2H₂O) was added to the resulting aqueous solution, stirred for 4 hours, centrifuged, washed three times with methanol, and then dried in vacuo at a temperature of 100° C. to obtain ZIF-11 powder.

### <Comparative Example 5> Preparation of ZIF-7-III

0.5 g of ZIF-11 obtained in Comparative Example 4 was immersed in 1 M KOH aqueous solution in an autoclave and then stirred under a condition at a speed of 500 rpm for 8 hours while being heated at a temperature of 150° C. The cooled aqueous solution was filtered, washed, and dried in vacuo at a temperature of 100° C. to obtain ZIF-7-III.

### <Comparative Example 6> Preparation of ZIF-8

A solution in which 284 mmol of 2-methylimidazole dissolved in 20 mol of methanol and a solution in which 34 mmol of a zinc nitrate hydrate (Zn(NO 3)₂·6H₂O) dissolved in 20 mol of methanol were prepared. Subsequently, the zinc solution was added to the 2-methylimidazole solution, stirred at room temperature for 2 hours, and left still for 12 hours. The obtained mixture was centrifuged, washed twice with methanol, and then dried at a temperature of 100° C. to obtain ZIF-8 powder.

### <Example 1> Preparation of Ir—NHC-ZIF A (INZ-A)

2.5 mol % of Ir-NHC prepared in Comparative Example 1, with respect to the number of moles of Zn contained in ZIF, dissolved in toluene. Then, the resulting product was additionally introduced into the composition for synthesizing ZIF-11, obtained in Comparative Example 4. Next, Ir-NHC@ZIF-11 powder in which Ir-NHC was entrapped in ZIF-11 was prepared in the same manner as the preparation method of ZIF-11, performed in Comparative Example 4.

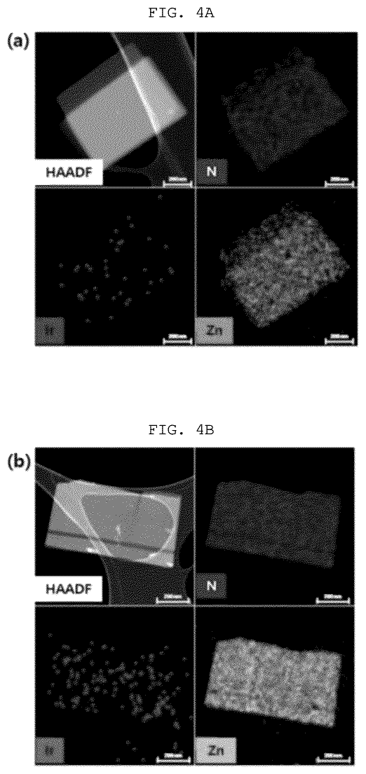

Thereafter, a coordination complex compound-ZIF complex A (INZ-A) was obtained in the same manner as in Comparative Example 5, except for using Ir-NHC@ZIF-11 instead of ZIF-11. The EDS mapping result of the obtained coordination complex compound-ZIF complex A (INZ-A) is shown in FIG. 4A.

### <Examples 2 and 3> Preparation of Ir—NHC-ZIF A (INZ-A) with Increased Amount of Introduced Coordination Complex Compound

A coordination complex compound-ZIF complex A (INZ-A) was obtained in the same manner as in Example 1, except for using each composition for synthesizing ZIF-11, which contained 5 mol % (Example 2) or 7.5 mol % (Example 3) of Ir-NHC prepared in Comparative Example 1, with respect to the number of moles of Zn contained in the ZIF.

### <Example 4> Preparation of Ir—NHC-ZIF B (INZ-B)

A coordination complex compound-ZIF complex B (INZ-B) was obtained in the same manner as in Comparative Example 5, except for introducing 0.5 mol % of Ir-NHC prepared in Comparative Example 1, with respect to the number of moles of Zn contained in the ZIF, into 0.5 g (1.67 mmol) of ZIF-11. The EDS mapping result of the obtained coordination complex compound-ZIF complex B (INZ-B) is shown in FIG. 4B.

### <Examples 5 and 6> Preparation of Ir—NHC-ZIF B (INZ-B) with Increased Amount of Introduced Coordination Complex Compound

A coordination complex compound-ZIF complex B (INZ-B) was obtained in the same manner as in Example 4, except for additionally introducing 1.0 mol % (Example 5), increased by 0.5 mol %, or 1.5 mol % (Example 6) of Ir-NHC prepared in Comparative Example 1, with respect to the number of moles of Zn contained in the ZIF, into 0.5 g (1.67 mmol) of ZIF-11.

### <Example 7> Preparation of Ir—NHC-ZIF C (INZ-C)

A coordination complex compound-ZIF complex C (INZ-C) was obtained in the same manner as in Example 4, except for introducing 0.5 mol % of Ir-NHC prepared in Comparative Example 1, with respect to the number of moles of Zn, into 0.5 g (1.67 mmol) of ZIF-7-III prepared in Comparative Example 5, instead of ZIF-11.

### <Example 8> Preparation of Ir—NHC-ZIF C (INZ-C) with Increased Amount of Introduced Coordination Complex Compound

A coordination complex compound-ZIF complex C (INZ-C) was obtained in the same manner as in Example 7, except for introducing 1.5 mol % of Ir-NHC prepared in Comparative Example 1, increased from 0.5 mol %, with respect to the number of moles of Zn.

### <Example 9> Preparation of Ir-NHC2-ZIF A (INZ2-A)

Ir-NHC2@ZIF-11 powder in which Ir-NHC2 was entrapped in ZIF-11 was prepared in the same manner as the preparation method of Ir-NHC@ZIF-11 performed, using Ir-NHC2 prepared in Comparative Example 2, instead of Ir-NHC.

Next, INZ2-A was obtained in the same manner as in Example 1, except for using Ir-NHC2@ZIF-11 instead of Ir-NHC@ZIF-11.

### <Example 10> Preparation of Ir-NHC2-ZIF B (INZ2-B)

INZ2-B was obtained in the same manner as in Example 4, except for introducing 0.5 mol % of Ir-NHC2 prepared in Comparative Example 2, with respect to the number of moles of Zn contained in the ZIF, into 0.5 g (1.67 mmol) of ZIF-11.

### <Example 11> Preparation of Ir-Cp*-ZIF (Ir-Cp*-B)

Ir-Cp*-B was obtained in the same manner as in Example 4, except for introducing 0.5 mol % of Ir-Cp* prepared in Comparative Example 3, with respect to the number of moles of Zn contained in the ZIF, into 0.5 g (1.67 mmol) of ZIF-11.

### <Example 12> Preparation of INZ-8

INZ-8 in which an Ir-NHC complex compound was fixed to a metal vacancy site of ZIF-8 was prepared in the same manner as in Comparative Example 6, except for introducing 0.5 mol % of Ir-NHC prepared in Comparative Example 1, with respect to the number of moles of Zn contained in the ZIF, into the 2-methylimidazole solution of Comparative Example 6.

### Experimental Example 1: Immobilization Efficiency (IE) Comparison Experiment According to Metal ICP Analysis in Catalyst

TABLE 1

| | | | Amount of | | | |
|---|---|---|---|---|---|---|
| | | | introduced | | | |
| | | | coordination | | | |
| | | | complex compound | | | |
| | | | with respect to | Zn | Ir | IE |
| No. | Example No. | Catalyst | Zn (mol %) | (%) | (%) | (%) |
| 1 | Comparative | — | N/A | 22.1 | — | — |
| | Example 2 | | | | | |
| 2 | Comparative | ZIF-7- | N/A | 22.0 | — | — |
| | Example 5 | III | | | | |
| 3 | Example 1 | INZ-A | 2.5 | 21.0 | 0.316 | 19.7 |
| 4 | Example 2 | INZ-A | 5.0 | 21.4 | 0.542 | 16.9 |
| 5 | Example 3 | INZ-A | 7.5 | 20.9 | 0.931 | 19.4 |
| 6 | Example 4 | INZ-B | 0.5 | 21.3 | 0.288 | 88.8 |
| 7 | Example 5 | INZ-B | 1.0 | 22.8 | 0.559 | 86.1 |
| 8 | Example 6 | INZ-B | 1.5 | 22.1 | 0.909 | 94.6 |
| 9 | Example 7 | INZ-C | 0.5 | 20.6 | 0.288 | 88.1 |
| 10 | Example 8 | INZ-C | 1.5 | 20.6 | 0.819 | 85.2 |
| 11 | Example 9 | INZ2-A | 2.5 | 21.6 | 0.0739 | 4.61 |
| 12 | Example 10 | INZ2-B | 0.5 | 20.5 | 0.328 | 98.2 |

According to Table 1, during ZIF-11 synthesis, the immobilization efficiency (hereinafter referred to as IE) values of the INZ-A complexes (Examples 1 to 3), in which the coordination complex compound Ir-NHC was introduced into the composition for synthesizing the ZIF, appeared to be less than 20%. On the other hand, in the case of the INZ-B complexes (Examples 4 to 6) in which the coordination complex compound was introduced during the phase transition process by heating the synthesized ZIF-11 and the INZ-C complexes (Examples 7 and 8) in which the coordination complex compound was introduced into ZIF-7-III, the IE values appeared to be improved by at least 4 times, compared to that of the INZ-A complexes. This is likely due to a large amount of Ir-NHC leached into the solution when preparing the Ir-NHC@ZIF-11 powder of Examples 1 to 3.

In addition, in the case of the INZ2-B complex (Example 10) in which the coordination complex compound was introduced during the phase transition process by heating the synthesized ZIF-11, the IE value appeared to be higher by 21 or more times than that of the INZ2-A complex (Example 9) in which the coordination complex compound Ir-NHC was introduced into the composition for synthesizing the ZIF during ZIF-11 synthesis.

FIG. 4A shows diagrams of EDS mapping results of coordination complex compound-ZIF complexes INZ-A of the present disclosure; and FIG. 4B shows diagrams of EDS mapping results of coordination complex compound-ZIF complexes INZ-B of the present disclosure; confirming that the active metal of the coordination complex compound, Ir, introduced into the ZIF was uniformly dispersed and supported.

### Experimental Example 2: Catalytic Activity Measurement Experiment in Glycerol Dehydrogenation

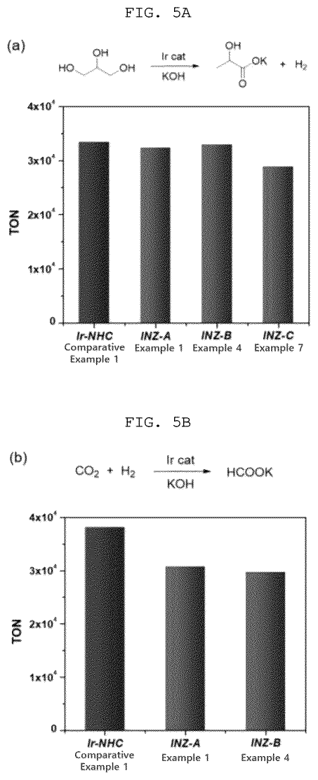

50 mL of a mixed solution of 0.5 umol of the catalyst (with respect to Ir), 50 mmol (50 mL) of 1 M glycerol, 55 mmol of 1.1 M KOH, and 44.492 mL of water was introduced into a 100-mL autoclave. Then, after raising a temperature inside the container to 180° C., the mixed solution was stirred at a speed of 500 rpm for 24 hours. Turnover number (TON) was calculated on the basis of the HPLC analysis results of lactic acid (LA) in the reaction product, using the following equation. The results thereof are shown in Table 2 below and FIG. 5A.

[Turnover Numbers (TONs) Calculation Equation]

TONs=(mmols of product)/(mmols of Ir in catalyst)

TABLE 2

| No. | Example No. | Catalyst | LA (mmol) | TON |
|---|---|---|---|---|
| 1 | — | — | 1.0 | — |
| 2^{[a]} | Comparative | ZIF-7-III | 2.0 | — |
| | Example 5 | | | |
| 3 | Comparative | Ir-NHC | 16.7 | 33,484 |
| | Example 1 | | | |
| 4 | Example 1 | INZ-A | 16.2 | 32,423 |
| 5 | Example 4 | INZ-B | 16.5 | 32,972 |
| 6 | Example 7 | INZ-C | 14.5 | 28,940 |
| 7 | Comparative | Ir-NHC2 | 12.6 | 25,249 |
| | Example 2 | | | |
| 8 | Example 9 | INZ2-A | 7.5 | 15,029 |
| 9 | Example 10 | INZ2-B | 7.7 | 15,430 |
| 10 | Comparative | Ir-Cp* | 10.1 | 20,100 |
| | Example 3 | | | |
| 11 | Example 11 | Ir-Cp*- B | 7.2 | 14,329 |
| 12^{[b]} | Example 1 | INZ-A | 18.4 | 36,826 |

* ^{[a]}using 50 mg of catalyst, ^{[b]}using 80% crude glycerol

According to Table 2, when using the coordination complex compound Ir-NHC catalyst, the homogeneous catalyst, alone, the TON value was the highest and the catalytic activity thus was at the highest level. In addition, compared to Comparative Example 1, in the case of the heterogeneous catalysts, INZ-A and INZ-B, the respective TON values of 97% and 98% were recorded, confirming that the catalytic performance remained nearly equivalent.

In addition, other coordination complex compounds, Ir-NHC2 (Comparative Example 2) and Ir-Cp* (Comparative Example 3), also showed activity in the corresponding reaction. Furthermore, in the case of the heterogeneous catalysts of Examples 9 to 11, INZ2-A, INZ2-B, and Ir-Cp*-B, TON values of about 60% and 71% were also recorded.

In addition, to confirm potential applicability in the industrial field, 80% crude glycerol was used instead of 1 M glycerol as the reactant in the presence of the INZ-A complex. As a result, an excellent TON value was recorded.

### Experimental Example 3: Catalytic Activity Comparison Experiment in CO₂ Hydrogenation

1 umol of the catalyst (with respect to Ir) was introduced into a high-pressure autoclave containing 50 mL of 1 M KOH and then purged with nitrogen. Next, after raising a temperature to 150° C. under the condition of introducing gas (CO₂:H₂=1:1) at a pressure of 60 bar, the resulting product was stirred at a speed of 500 rpm for 24 hours. TON was calculated on the basis of the HPLC analysis results of formic acid (FA) in the reaction product, using the above equation. The results thereof are shown in Table 3 below and FIG. 5B.

Table 3

| No. | Example No. | Catalyst | FA (mmol) | TON |
|---|---|---|---|---|
| 1 | — | — | 0.3 | — |
| 2^{[a]} | Comparative | ZIF-7-III | 6.3 | — |
| | Example 5 | | | |
| 3 | Comparative | Ir-NHC | 38.2 | 38,196 |
| | Example 1 | | | |
| 4 | Example 1 | INZ-A | 30.8 | 30,807 |
| 5 | Example 4 | INZ-B | 29.8 | 29,748 |

* ^{[a]}using 50 mg of catalyst

According to Table 3, when using the coordination complex compound the Ir-NHC catalyst alone in the CO₂ dehydrogenation, the activity was at the highest level. In addition, compared to Ir-NHC, in the case of the heterogeneous catalysts, INZ-A and INZ-B, the respective TON values of 81% and 78% were recorded, confirming that the catalyst performance was not significantly deteriorated.

### Experimental Example 4: High-Temperature Filtration Experiment of Complex Catalyst (INZ-A) of Example 1 in CO₂ Hydrogenation

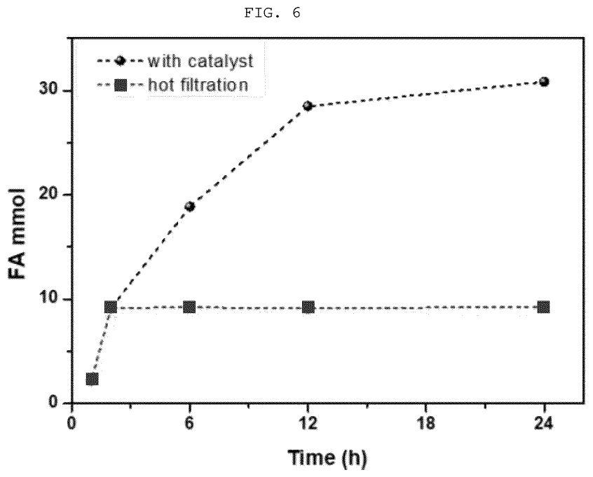

The CO₂ hydrogenation was performed in parallel in the same manner as in Experimental Example 3 using the same amount of the complex catalyst (INZ-A) prepared in Example 1. However, while one side reaction was not subjected to high-temperature filtration as a control group, in the other side reaction, high-temperature filtration was performed on the aqueous solution containing the catalyst after 2 hours of reaction time. The changes in the number of moles of formic acid (FA), which is the reaction product, are shown in FIG. 6.

Referring to FIG. 6, in the case where the catalyst was not subjected to high-temperature filtration in the reaction system for a reaction time of 0 to 24 hours, FA was found to be continuously produced. On the contrary, in the case of performing high-temperature filtration, after the reaction time of 2 hours, the heterogeneous catalyst of Example 1 was confirmed to be removed, so the number of moles of FA, the reaction product, was not increased.

### Experimental Example 5: Catalytic Activity Measurement Experiment in K₂CO₃-Mediated Transfer-Hydrogenation of Glycerol

50 mL of a mixed solution of 3 μmol of the catalyst (with respect to Ir), 200 mmol of 4 M glycerol, 100 mmol of 2 M K₂CO₃, and 35.38 mL of water was introduced into a 200-mL high-pressure autoclave reactor and then purged with nitrogen. Next, after raising a temperature to 180° C. under the condition of introducing nitrogen gas at a pressure of 26 bar, the resulting product was stirred at a speed of 500 rpm for 3 to 24 hours. TON was calculated on the basis of the HPLC analysis results of formic acid (FA) and lactic acid (LA) in the reaction product, using the above equation. The results thereof are shown in Table 4 below.

TABLE 4

| | | | LA/FA | |
|---|---|---|---|---|
| No. | Example No. | Catalyst | (mmol) | TON |
| 1^{[a]} | Comparative | ZIF-7-III | 7.7/1.1 | — |
| | Example 5 | | | |
| 2 | Comparative | Ir-NHC | 50.3/11.4 | 15,076/3,447 |
| | Example 1 | | | |
| 3 | Example 1 | INZ-A | 45.0/10.6 | 13,514/3,191 |
| 4 | Example 4 | INZ-B | 46.2/10.0 | 13,887/3,009 |

* ^{[a]}using 200 mg of catalyst

According to Table 4, in the transfer-hydrogenation of glycerol, compared to the homogeneous catalyst, Ir-NHC, in the case of the heterogeneous catalysts, INZ-A and INZ-B, the respective TON values of 90% and 92%, based on lactic acid (LA), were recorded, and the respective TON values of 93% and 87%, based on formic acid (FA), were recorded. As a result, the catalyst performance of the heterogeneous catalysts was confirmed to be not significantly deteriorated compared to that of the homogeneous catalyst.

### Experimental Example 6: Activity Measurement Experiment of INZ-A Complex Catalyst Reused for K₂CO₃-Mediated Transfer-Hydrogenation of Glycerol

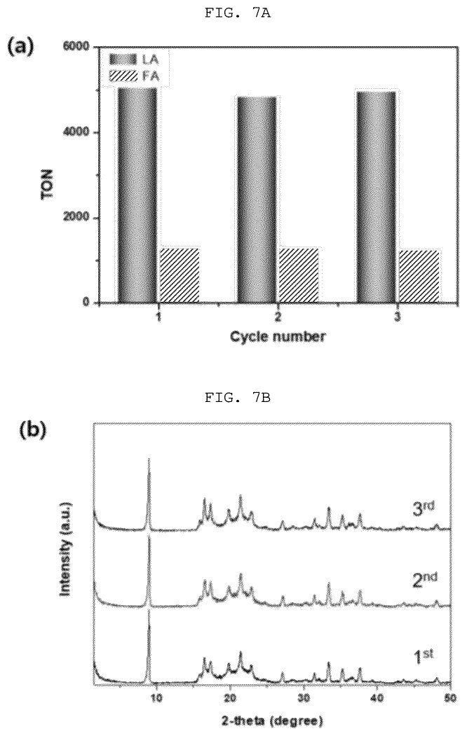

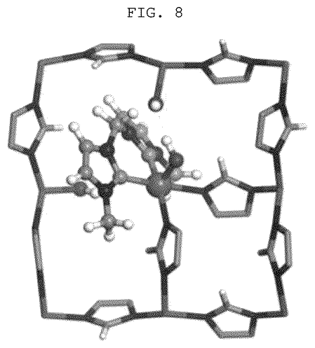

The complex catalyst (INZ-A) of Example 1 used in the K₂CO₃-mediated transfer-hydrogenation of glycerol of Experimental Example 5 was recovered and reused 1 to 3 times. The TON (Turnover Number) measurement results are shown in FIG. 7A, and the PXRD measurement results are shown in FIG. 7B.

Referring to FIG. 7A and FIG. 7B, even though the number of reuses was accumulated up to 3 times, the TON value remained nearly equivalent. In addition, in the XRD pattern measurement results, the crystallinity well remained without change, and it was confirmed that there was no ICP-caused Ir leaching (the ICP result is not shown).

Although the present disclosure has been described above with reference to the embodiments described herein or shown in the accompanying drawings, these embodiments are disclosed for illustrative purposes. Those skilled in the art will appreciate that various modifications, additions, and substitutions are possible, without departing from the scope and spirit of the invention as disclosed in the accompanying claims. Therefore, the scope of protection of the present disclosure should be defined by the appended claims.

What is claimed is:

1. A method of preparing a coordination complex compound-ZIF complex, the method comprising:
(a) preparing a coordination complex compound precursor comprising both of a ligand more strongly bonded to a central metal of a coordination complex compound than imidazolate and a ligand more weakly bonded to the central metal of the coordination complex compound than imidazolate; and
(b) obtaining a coordination complex compound-ZIF complex by adding the coordination complex compound precursor and a ZIF to a basic solution and heating the resulting solution to a temperature in a range of 5° C. to 250° C.

2. A method of preparing a coordination complex compound-ZIF complex, the method comprising:
(A) preparing a coordination complex compound precursor and a composition for synthesizing a ZIF, the coordination complex compound precursor comprising both of a ligand more strongly bonded to a central metal of a coordination complex compound than imidazolate and a ligand more weakly bonded to the central metal of the coordination complex compound than imidazolate; and
(B) obtaining a coordination complex compound-ZIF complex by heating a mixed solution of the coordination complex compound precursor, the ZIF, and a base to a temperature in a range of 5° C. to 250° C.

3. The method of claim 1, wherein the ZIF is one of ZIF-11, ZIF-1, ZIF-4, ZIF-7, ZIF-8, ZIF-9, ZIF-12, ZIF-35, ZIF-67, ZIF-69, ZIF-71, ZIF-90, ZIF-95, and ZIF-100.

4. The method of claim 1, wherein the coordination complex compound is added in an amount in a range of 0.01 to 10 mol % with respect to the number of moles of Zn contained in the ZIF.

5. A complex in which a coordination complex compound is bonded to a ZIF, wherein a central metal of the coordination complex compound is chemically and directly bonded to a metal vacancy site of the ZIF.

6. The complex of claim 5, wherein a ligand of the coordination complex compound is more strongly bonded to the central metal of the coordination complex compound than imidazolate, thereby being unsubstituted by the imidazolate.

7. The complex of claim 5, wherein the ZIF is one of ZIF-11, ZIF-1, ZIF-4, ZIF-7, ZIF-8, ZIF-9, ZIF-12, ZIF-35, ZIF-67, ZIF-69, ZIF-71, ZIF-90, ZIF-95, and ZIF-100.

8. The complex of claim 5, wherein the central metal of the coordination complex compound comprises at least one of iridium (Ir), cobalt (Co), iron (Fe), ruthenium (Ru), rhodium (Rh), platinum (Pt), palladium (Pd), gold (Au), silver (Ag), copper (Cu), nickel (Ni), titanium (Ti), zirconium (Zr), chromium (Cr), molybdenum (Mo), and rhenium (Re).

9. The complex of claim 5, wherein a ligand of the coordination complex compound comprises at least one of carbene, phosphine, amine, and cyclopentadienyl.

10. The complex of claim 5, wherein the coordination complex compound-ZIF complex is represented by Formula 1,
M₁₋ₓ(L)₂(MC)ₓ [Formula 1]
(wherein in Formula 1, M is the central metal of the ZIF and is selected from among Zn, Co, Ni, Fe, Mn and Pd,
L is imidazolate,
MC is the coordination complex compound chemically bonded to a metal vacancy site of the ZIF, wherein a ligand bonded to the central metal of the coordination complex compound is more strongly bonded to the central metal of the coordination complex compound than imidazolate, thereby being unsubstituted by the imidazolate, and
x is a real number greater than 0 and less than or equal to 0.1).

11. A method of obtaining a conversion product of a hydrocarbon containing at least one OH group by transfer-hydrogenation of the hydrocarbon, using a catalyst comprising the complex of claim 5.

12. A method of obtaining formic acid by converting carbon dioxide, using a catalyst comprising the complex of claim 5.
